(19) United States
(12) Patent Application Publication
(10) Pub. No.: US 2023/0190977 A1
(43) Pub. Date: Jun. 22, 2023

(54) AN AUGMENTED REALITY DEVICE FOR DETERMINING DISINFECTION LEVELS CAUSED BY LIGHTING DEVICE AND A METHOD THEREOF

(71) Applicant: SIGNIFY HOLDING B.V., EINDHOVEN (NL)

(72) Inventors: NAM CHIN CHO, PEACHTREE CITY, GA (US); PETER DEIXLER, ARLINGTON, MA (US); PARTH JOSHI, ATLANTA, GA (US)

(21) Appl. No.: 17/926,342

(22) PCT Filed: May 20, 2021
(86) PCT No.: PCT/EP2021/063484

§ 371 Date: Nov. 18, 2022

Related U.S. Application Data
(60) Provisional application No. 63/031,105, filed on May 28, 2020.

(30) Foreign Application Priority Data
Jun. 9, 2020 (EP) 20178858.5

(51) Int. Cl. A61L 2/28; A61L 2/24; A61L 2/10; G06T 19/00

(52) U.S. Cl. CPC A61L 2/28; A61L 2/10; A61L 2/24; G06T 19/006; A61L 2202/14; A61L 2202/25

(57) ABSTRACT

A method (600) of determining disinfection levels of a lighting device (130) is disclosed. The lighting device (130) is configured to emit disinfection light. The method (600) comprises: capturing (602), by a camera 108 of an augmented reality device (102), an image of a physical area (100), displaying (604), on a display (110) of the augmented reality device (102), the image, determining (606) an irradiation value of disinfection light provided or to be provided by the lighting device 130 on the target surface (140), wherein the irradiation value is determined based on a location of the lighting device (130) with respect to the target surface (140) and based on properties of the disinfection light provided or to be provided by the lighting device (130), determining (608) a level of disinfection of the target surface (140) based on the irradiation value of disinfection light, and rendering (610), on the display (110) of the augmented reality device (102), an indicator (120) indicating the level of disinfection, wherein the indicator (120) is overlaid on the image of the physical area (100).

### FIELD OF THE INVENTION

The invention relates to a method of determining disinfection levels caused by a lighting device and to a computer program product for executing the method.

The invention further relates to an augmented reality device for determining disinfection levels caused by a lighting device.

### BACKGROUND

Disinfection light is being used for disinfecting surfaces in different types of locations, such as homes, public spaces, hospitals, etc. A general advantage of light-based disinfection is that the disinfection is not surface specific but works on air, hard and soft surfaces and generally on all places where the light can reach the germs. Ultraviolet (UV) light may be used for disinfection purposes, for instance for disinfecting air, water or medical instruments. However, over-exposure to UV light, in particular UV-B and certain UV-C wavelengths, provides health risks for living beings and thus has to be avoided.

UV lighting is divided into three types. UV-A (400-315 nm), UV-B (315-280) and UV-C (100-280 nm). UV-C light is effective to disinfect surface from pathogens like bacteria, viruses and molds. To effectively disinfect surfaces with UV-B or UV-A light longer exposure times or greater light intensities may be required. For instance, UV-A disinfection may require irradiance levels as high as 3 W m² at the target surface. Recent developments have shown that alternative to UV light, also light with longer wavelengths, in particular light in the visible spectrum around 405 nm, can be used for disinfection purposes, since this light is absorbed by porphyrin molecules inside bacteria, causing the formation of biocidal reactive oxygen. This process allows to suffocate and kill the bacteria and can lead to a reduced contamination when applied continuously to an area. Moreover, such light disinfection can also be advantageous for food processing facilities or agriculture and horticulture applications.

For different locations different disinfection levels are required. For instance, in public spaces the required level of disinfection may be higher than in homes. A lighting installer should therefore select the correct type and number UV lighting devices for the location, and configure any installed lighting devices accordingly.

WO 2019079065 A1 discloses method for aiding disinfection of a room. The method may include collecting, by one or more sensors in a disinfection system, activity data in the room. A computing device or output device may identify one or more hot spots from the activity data, in which the one or more hot spots indicate areas in the room for cleaning, and generate a contamination map containing the one or more hot spots. The output device may output the contamination map to an output device for viewing by a user. The output device may be an augmented reality system that overlays the contamination map onto a person’s view of the room. The output device may include goggles for the user to wear. The goggles may overlay or project a virtual image of the hot spots onto a view of the room that the user sees through the goggles. Thus the output device indicates to the user which areas of the room are considered hot spots that should be cleaned.

### SUMMARY OF THE INVENTION

The inventors have realized that it may be difficult for certain users to select and/or configure a disinfection lighting device, because it is not clear what the disinfection effect in a physical area may be. It is therefore an object of the present invention to provide a method to support a user in selecting and/or configuring a disinfection lighting device.

According to a first aspect of the present invention, the object is achieved by a method of determining disinfection levels caused by a lighting device installed in or to be installed in a physical area, the lighting device being configured to emit disinfection light, the method comprising:
capturing, by a camera of an augmented reality device, an image of a physical area,
displaying, on a display of the augmented reality device, the image,
determining an irradiation value of disinfection light provided or to be provided by the lighting device on the target surface, wherein the irradiation value is determined based on a location of the lighting device with respect to the target surface and based on properties of the disinfection light provided or to be provided by the lighting device,
determining a level of disinfection of the target surface based on the irradiation value of disinfection light, and
rendering, on the display of the augmented reality device, an indicator indicating the level of disinfection, wherein the indicator is overlaid on the image of the physical area.

The lighting device may be a lighting device has already been installed in the physical area or the lighting device may be a lighting device that still has to be installed in the physical area. By determining the irradiation value for the target surface in the physical area, the level of disinfection of the target surface by the lighting device can be determined. The irradiation value, and therewith the level of disinfection, are at least dependent on the location of the lighting device with respect to the target surface, and on properties (e.g. intensity, beam shape, intensity distribution within the beam, beam direction, etc.) of the disinfection light (e.g. ultraviolet or infrared light) provided or to be provided by the lighting device. By rendering an indicator indicating the level of disinfection as an overlay on the image of the physical area, the user can immediately see the effect of the lighting device on the target surface in the physical area. This is beneficial, because it may support a user in selecting a certain disinfection lighting device (such as UV LED, mercury lamps, xenon lamps and excimer lamps) and/or configuring a UV lighting device. If the lighting device is yet to be installed, the user may for example decide to select a certain lighting device and see its disinfection level, which supports the user in selecting the correct lighting device for the physical area. Alternatively, if the lighting device has already been installed/positioned in the physical area, the user may for example decide to change the lighting device’s light emission properties and/or reposition the device based on the indicated disinfection level, which supports the user in configuring the lighting device.

The lighting device may have been installed in the physical area. Configuring a disinfection lighting device may be harmful to a user’s skin or eyes when the disinfection lighting device is switched on. Therefore, it may be beneficial to switch off or dim the light output of the lighting device below a threshold dimming level during configuration, and use augmented reality to provide the disinfection level/a possible disinfection level of the lighting device.

The beam of disinfection light of the lighting device may be adjustable. The method may further comprise: receiving input indicative of an adjustment of the beam of disinfection light of the lighting device, and adjusting the beam of disinfection light of the lighting device based on the input. The shape and/or the direction of the beam may be adjustable. The input may, for example, be a user input, enabling a user to change the disinfection level of the target surface. Alternatively, the input may be a system input for automatic adjustment of the beam, for instance to automatically increase or decrease the disinfection level of the target surface.

The lighting device may be a to-be-installed lighting device, and the method may further comprise:
receiving an input indicative of a position of the to-be-installed lighting device with respect to the physical area,
rendering, on the display of the augmented reality device, a virtual representation of the to-be-installed lighting device, wherein the virtual representation is overlaid on the image of the physical area at the position. The virtual representation represents the to-be-installed lighting device. The input may, for example, be a user input. The user may position the virtual representation on the image, whereupon the level of disinfection of the target surface may be determined based on the (new) position/location of the lighting device relative to the target surface. Alternatively, the input may be a system input for automatic placement of the lighting device, for instance at a central location in the physical area, or above a predefined target surface in the physical area.

The method may further comprise the step of selecting the lighting device from a plurality of lighting devices based on one or more disinfection criteria. The method may further comprise: receiving user input indicative of the one or more disinfection criteria. Additionally or alternatively, the one or more disinfection criteria may be predefined and/or determined based on information of the physical area. This information may be extracted from the image and/or may be retrieved from a memory. Examples of disinfection criteria include but are not limited to: a disinfection level per surface area, information about the type of material of the target surface, a type of physical area and/or target surface, activities typically performed in the physical area/at the target surface, a type of pathogen of which the target surface needs to be disinfected, a degradation characteristics of the surface under disinfection lighting exposure, power consumption of the disinfection, etc.

The method may further comprise:
obtaining contextual parameters about the physical area and/or the target surface, and
determining the level of disinfection further based on the contextual parameters. The method may further comprise: receiving user input indicative of the contextual parameters. Additionally or alternatively, the contextual parameters may be predefined and/or determined based on information of the physical area. The contextual parameters may be extracted from the image and/or may be retrieved from a memory.

Examples of contextual parameters include but are not limited to: a type of physical area and/or target surface, activities typically performed in the physical area/at the target surface, information about the type of material of the target surface, etc.

The method may further comprise:
determining a second irradiation value of disinfection light provided or to be provided by the lighting device on a second target surface in the physical area, wherein the second irradiation value is determined based on the location of the lighting device with respect to the second target surface and based on properties of the disinfection light provided or to be provided by the lighting device,
determining a second level of disinfection of the second target surface based on the second irradiation value of disinfection light, and
rendering, on the display of the augmented reality device, a second indicator indicating the second level of disinfection, wherein the second indicator is overlaid on the image of the physical area. It may be beneficial to distinguish between different target surfaces (e.g. a floor and a table) and to indicate the different levels of disinfection of the respective target surfaces.

The method may further comprise:
determining a further irradiation value of disinfection light provided or to be provided by a further lighting device on the target surface, wherein the further lighting device has been installed in the physical area, and wherein the further irradiation value is determined based on a location of the further lighting device with respect to the target surface and based on properties of the disinfection light provided or to be provided by the further lighting device,
determining a further level of disinfection of the target surface based on the further irradiation value of disinfection light, and
rendering, on the display of the augmented reality device, a further indicator indicating the further level of disinfection, wherein the further indicator is overlaid on the image of the physical area. It may be beneficial to determine different levels of disinfection of different lamps on the target surface and to visualize those as an overlay on the image. The indicators of the different levels of disinfection may be rendered such that they are distinguishable by a user, which is beneficial because the effect of disinfection of both lighting devices on the target surface is clear to the user.

The method may further comprise:
determining a period of time that a person, animal or an object can safely be exposed to the disinfection light, and
rendering a safety indicator indicative of the period of time. If the user would change one of the properties of the UV light, the safety indicator may change. This indicates to a user how long that user can safely be exposed to the UV light.

The method may further comprise: rendering, on the display of the augmented reality device, a beam shape indicator overlaid on the image of the physical area, wherein the beam shape indicator indicates the shape of the beam of the disinfection light. This is beneficial, because the user can immediately see the area illuminated by the UV light source.

The method may further comprise:
receiving user input indicative of an adjustment of the properties of the disinfection light provided or to be provided by the lighting device, and
adjusting the properties of the disinfection light based on the user input. The user may provide input to adjust the properties (e.g. the intensity or spectrum of the light, etc.), whereupon the irradiation value, and therewith the level of disinfection, may be determined based on the adjusted properties. This is beneficial, because any changes made to the light output of the lighting device are reflected on the display.

According to a second aspect of the present invention, the object is achieved by a computer program product for a computing device, the computer program product comprising computer program code to perform any of the above-mentioned methods when the computer program product is run on a processing unit of the computing device.

According to a third aspect of the present invention, the object is achieved by an augmented reality device for determining disinfection levels caused by a lighting device installed in or to be installed in a physical area, the lighting device being configured to emit disinfection light, the augmented reality device comprising:
a camera configured to capture an image of a physical area,
a display configured to display the image,
a processor configured to:
   determine an irradiation value of disinfection light provided or to be provided by the lighting device on the target surface, wherein the irradiation value is determined based on a location of the lighting device with respect to the target surface and based on properties of the disinfection light provided or to be provided by the lighting device, determine a level of disinfection of the target surface based on the irradiation value of disinfection light, and to
   render, on the display, an indicator indicating the level of disinfection, wherein the indicator is overlaid on the image of the physical area.

It should be understood that the computer program product and the augmented reality device may have similar and/or identical embodiments and advantages as the above-mentioned methods.

### BRIEF DESCRIPTION OF THE DRAWINGS

The above, as well as additional objects, features and advantages of the disclosed systems, devices and methods will be better understood through the following illustrative and non-limiting detailed description of embodiments of devices and methods, with reference to the appended drawings, in which:

FIG. 1 shows schematically an embodiment of an augmented reality device for determining disinfection levels caused by a lighting device in a space;

FIG. 2 shows schematically an embodiment of an augmented reality device rendering disinfection levels caused by a lighting device in a space;

FIG. 3 shows schematically an embodiment of an augmented reality device rendering disinfection levels of different surfaces in a space;

FIGS. 4a and 4b show schematically an augmented reality device for receiving a user input indicative of an adjustment of a beam of light caused by a lighting device;

FIG. 5 shows an example of a software application running on an augmented reality device; and

FIG. 6 shows schematically a method of determining disinfection levels caused by a lighting device.

All the figures are schematic, not necessarily to scale, and generally only show parts which are necessary in order to elucidate the invention, wherein other parts may be omitted or merely suggested.

### DETAILED DESCRIPTION OF EMBODIMENTS

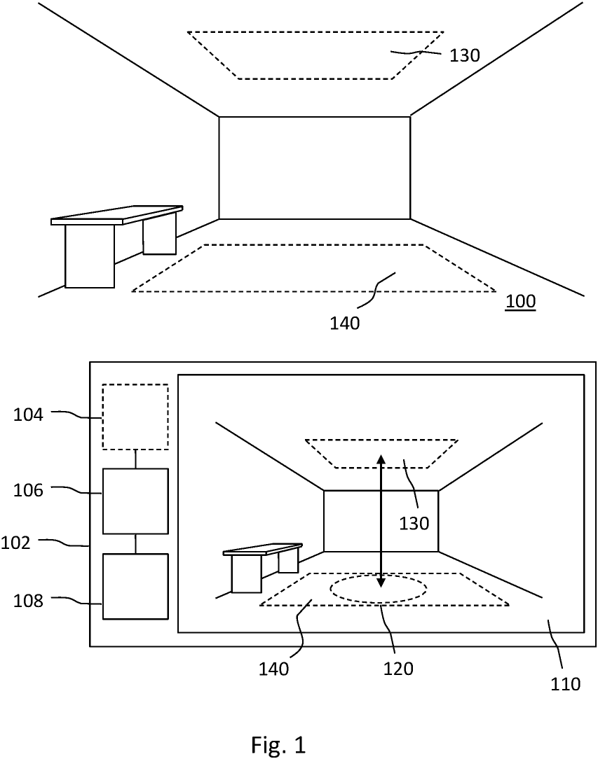

FIG. 1 shows schematically an embodiment of an augmented reality device 102 for determining disinfection levels caused by a lighting device 130 in a physical area 100 (e.g. a room). The augmented reality device 102 comprises a camera 108 for capturing images of a space, a display 110 configured to display the images captured by the camera 108 and a processor 106. The augmented reality device 102 may, for example, be a smartphone, smart glasses, a tablet pc, etc. The processor 106 may render the captured image on the display 110. The image comprises the lighting device 130. The image may be a real-time image which enables a user to move the augmented reality device 102 around to capture different parts of the physical area 100 (e.g. the room).

The processor 106 (e.g. one or more microprocessors and/or microcontrollers) is configured to determine an irradiation value (e.g. an irradiation level, the lux, the intensity, etc.) of disinfection light provided (when the lighting device 130 has already been installed and is switched on) or to be provided (when the lighting device 130 is a to-be-installed lighting device or when the lighting device 130 has already been installed but is switched off) by the lighting device 130 on the target surface 140. The processor 106 is configured to determine the irradiation value based on a location of the lighting device 130 with respect to the target surface 140 and based on properties (e.g. light emission characteristics) of the disinfection light provided or to be provided by the lighting device 130. The augmented reality device 102 may comprise a lighting design software application and a database of lighting devices along with associated photometric files or data files of lighting information. The processor 106 may be configured to use a photometric file of the lighting device to estimate one or more lighting patterns that can be produced by lighting devices within a three-dimensional space (e.g. the physical area 100). A photometric file may include information related to light properties of the lighting devices, such as beam shape, beam width, light intensity, etc. The photometric data file may be an Illuminating Engineering Society (IES) file or another photometric data file. Lighting data that is provided to the augmented reality device 102 by a user may be used instead of or in addition to the photometric data. The processor 106 may use the lighting data/photometric data to determine the irradiation value of the target surface 140.

The processor 106 may be further configured to obtain an identifier of the lighting device 130 and determine the light properties (beam shape, beam width, light intensity, etc.) of the disinfection light provided or to be provided by the lighting device 130. The processor 106 may identify the lighting device 130, for example based on a code emitted by the lighting device 130, the code being indicative of the identifier, based on a user input indicative of the identifier, based on an identifier received from a central lighting control system, etc. Techniques for identifying a lighting device 130 that has been installed in a physical area 100 are known in the art and will therefore not be discussed in further detail.

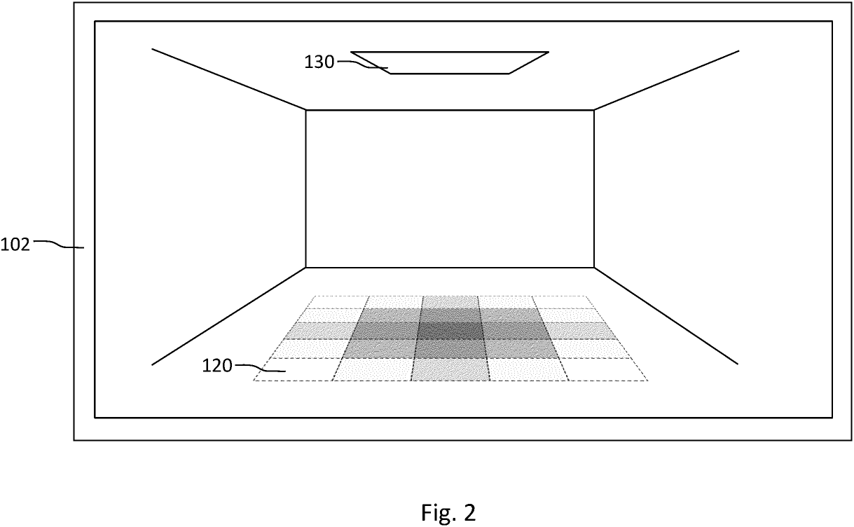

The processor 106 is further configured to determine a level of disinfection of the target surface 140 based on the irradiation value of disinfection light. The level of disinfection is dependent on the type of UV light that is used (i.e. the spectrum of the light) (UV-A, UV-B or UV-C) and the intensity of the disinfection light. For instance, UV-C is more effective killing pathogens compared to UV-B. To effectively disinfect surfaces with UV-B or UV-A light longer exposure times may be required. The level of disinfection may indicate to what extent the target surface 140 is disinfected by the disinfection light that is emitted by the lighting device. The processor 106 is further configured to render, on the display, an indicator 120 indicating the level of disinfection, wherein the indicator 120 is overlaid on the image of the physical area 100. The indicator may, for example, indicate the level of disinfection for different subareas of the target surface (see FIG. 2), may indicate a duration/exposure time required to disinfect the target surface from a certain pathogen for the targeted log reduction (i.e. a relative number of living microbes that are eliminated by disinfection, e.g. a 1 log reduction corresponds to inactivating 90 percent of a target microbe with the microbe count being reduced by a factor of 10), may indicate which areas are safe/unsafe for a human being to be exposed to, may indicate the power consumption of the lighting device 130 for disinfection, etc.

The level of disinfection may be indicative of an exposure time required to disinfect the target surface 140, and the processor 106 may render the indicator such that it indicates the exposure time. The required exposure time is the time required to disinfect the target surface 140 with the disinfection light provided or to be provided by the lighting device, and the processor 106 may determine the exposure time dependent on the irradiation value of the lighting device 130. The processor 106 may determine the exposure time further based on one or more contextual parameters, such as a type of physical area 100 and/or target surface 140, activities typically performed in the physical area 100 /at the target surface 140, information about the type of material of the target surface 140, a type of pathogen, etc. For example, the darker gray areas in FIGS. 2-5 may be indicative of a shorter exposure time required to disinfect the target surface 140 (e.g. for a maximum, user-defined or predefined irradiation value of the light emitted by the lighting device 130), and the light gray areas in FIGS. 2-5 may be indicative of a longer exposure time required to disinfect the target surface 140.

The lighting device 130 is configured to emit disinfection light. The lighting device may be a luminaire already installed or to-be-installed in the space. The lighting device 130 is adapted to emit light in a range of the electromagnetic spectrum, wherein at least a part of the light spectrum emitted by the lighting device 130 is usable for disinfection purposes. The lighting device 130 may be adapted to emit at least disinfection light in the deep blue and/or purple part of the visible spectrum, for instance comprising a wavelength of 405 nm. Additionally or alternatively, the lighting device can also be adapted to emit disinfection light in an ultraviolet part of the spectrum, preferably in the UV-B and more preferably in the UV-C part of the spectrum. Moreover, also light in the infrared spectrum can be employed for disinfection purposes, with respect to certain pathogens.

The lighting device 130 may be adapted to emit only the disinfection light. Alternatively, the lighting device 130 may be adapted to emit light in addition to the disinfection light, for instance in a visible part of the electromagnetic spectrum and/or in the infrared part of the electromagnetic spectrum.

Different pathogens vary in UV susceptibility, some being relatively harder to kill than others. Shorter-wavelength UV photons have higher energy potential than longer-wavelength UV photons, and may have an accelerated aging effect on materials and paints. The required spectrum to be provided by the lighting device 130 and used for disinfection purposes may therefore depend on contextual parameters and/or disinfection criteria, such as materials present in the room, the material of the object to be disinfected, the presence of living beings in a room, the pathogen to be removed, etc.

The light may be provided such that the object is exposed to radiation varying from 200 to 1,000 J/m², i.e. 20 to 100 mJ/cm², depending on the type of surface and its cleanliness. The lighting device 130 may comprise one or more light sources, for instance one or more light emitting diodes (LEDs), low-pressure mercury germicidal lamps, pulsed xenon arc germicidal ultraviolet irradiation lamps or rare gas-halogen such as krypton-chlorine discharge light sources, etc. Additionally or alternatively, the lighting device 130 may comprise other light providing devices like a VCSEL, LASER, or a gas discharge lamp.

The (UV) disinfection light may be embedded in a retrofit downlight which provides light with a wavelength of both 222 nm Far-UV-C for disinfection purposes and general illumination. Such downlights can be applied to target disinfection applications in hospitals, clinics, assisted living communities, as well as schools & childcare centers.

The lighting device 130 may comprise different light source technologies, for instance a 222 nm excimer Kr-Cl lamp for virus disinfection in presence of people, a 256 nm mercury bulb providing more energy-efficient disinfection if the room is vacant and/or an UV-A LED anti-bacterial light source or purple LED for visible disinfection.

The lighting device 130 may comprise multiple light sources that are subsequently switched on/off to provide the disinfectant light to the target surface 140. The multiple light sources may be directed in different (adjustable) directions.

The processor 106 may be configured to determine irradiation values for various subareas on the surface 140 (e.g. the ground) based on the location of the lighting device 130 relative to the target surface 140. This has been illustrated in FIG. 2, which shows an augmented reality device 102 rendering a plurality of indicators 120 (squares) on the target surface. The indicators 120 indicate the levels of disinfection for respective subareas. Different colors (shades of gray in FIG. 2) may indicate different levels of infection.

The processor 106 may further be configured to obtain or determine a 3D model of the physical area 100. The processor 106 may, for example, obtain a Building Information Model (BIM) of the physical area 100, or the processor 106 may determine the 3D model based on sensor data from various depth sensors, which may be comprised in the augmented reality device 102. The augmented reality device 102 may, for example, comprise one or more depth sensors/cameras to create a 3D map of the physical area 100. Techniques for creating a 3D map/model of a physical area are known in the art and will therefore not be discussed in detail. In embodiments wherein the lighting device 130 has already been installed in the physical area 100, the processor 106 may be further configured to determine the location of the lighting device 130 based on the obtained/determined model and/or based on location information, which may for example be received from an (indoor) localization system. In embodiments wherein the lighting device 130 is yet to be installed in the physical area 100, the lighting design application may enable a user to select and place one or more lighting devices in the image, whereupon the processor 106 may determine the disinfection level of the selected lighting device 130. Similarly, the processor 106 may be configured to obtain the orientation of the lighting device 130 with respect to the target surface 140, and the processor 106 may determine the irradiation value of the target surface 140 further based on the orientation. The orientation may be determined based on a user input indicating the orientation, based on sensor data of an orientation sensor comprised in the (already installed) lighting device 130, based on analysis of an image of the (already installed) lighting device 130, etc.

The target surface 140 may be defined in various ways. The processor 106 may be configured to receive a user input (e.g. via the user interface 104) indicative of the target surface 140. The user may, for example, indicate the target surface in the image by providing a touch input via the touch screen. Alternatively, the target surface 140 may be predefined and, for example, based on the beam properties of the lighting device 130. The target surface 140 may be defined by an area illuminated by the lighting device 130. The processor 106 may determine which area is/would be illuminated by the lighting device 130, and define the illuminated area as the target surface 140. The target surface 140 may be detected in one or more images captured by the camera 108 (e.g. by analyzing the image applying known image analysis techniques), or determined based on the 3D-model of the physical area 100. Alternatively, the target surface 140 may be a surface of a virtual object that has been overlaid on the image.

The augmented reality device 102 may comprise a user interface 104 configured to receive user input. The user interface 104 may, for example, be a touch screen for receiving touch input, a microphone for receiving audio input, a gesture sensor for receiving gesture input, etc. Alternatively, the user interface for receiving user input may be an auxiliary device such as a smart assistant, a handheld controller, a control glove, etc. The user interface 104 may be configured to receive various user inputs with different functions, as described below.

The augmented reality device 102 may further comprise a communication unit (not shown) configured to communicate with other devices, for example an already installed lighting device 130. The processor 106 may communicate lighting control commands to the lighting device 130 via the communication unit, for example to change a property of the UV light of the lighting device 130 (e.g. switching the lighting device 130 on or off, changing the light intensity, changing the beam shape/angle, etc.). The communication unit may comprise hardware for communicating via any communication protocol. Various communication protocols may be used, for example Bluetooth, Wi-Fi, Li-Fi, 3G, 4G, 5G or ZigBee.

The lighting device 130 may have already been installed in the physical area 100. Configuring a disinfection lighting device may be harmful to a user’s skin or eyes when the disinfection lighting device is switched on, especially for UV-B or UV-C light. Therefore, it may be beneficial to switch off or dim the light output of the lighting device 130 below a (safe) threshold dimming level during configuration, and use augmented reality to provide the disinfection level of the lighting device 130.

The beam of disinfection light of the lighting device may be adjustable. The method may further comprise: receiving input indicative of an adjustment of the beam of disinfection light of the lighting device, and adjusting the beam of disinfection light of the lighting device based on the input. The shape and/or the direction of the beam may be adjustable. The input may, for example, be a user input, enabling a user to change the disinfection level of the target surface. Alternatively, the input may be a system input for automatic adjustment of the beam, for instance to automatically increase or decrease the disinfection level of the target surface.

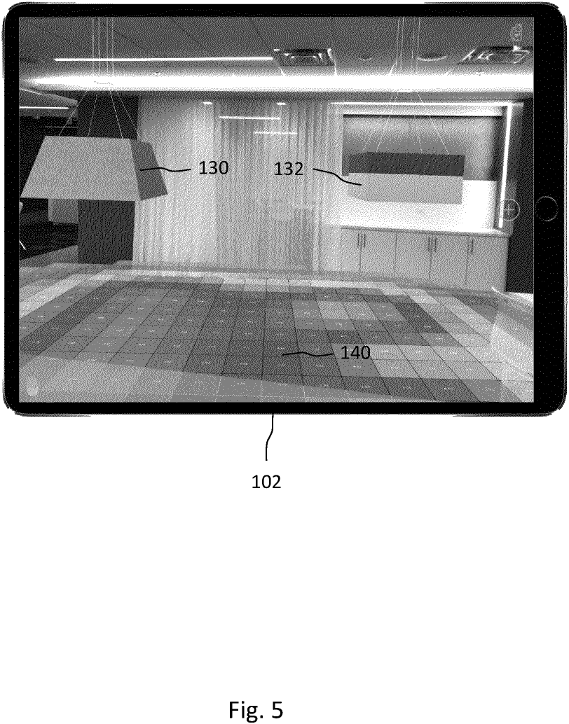

The lighting device 130 may be a to-be-installed lighting device. The processor 106 may be further configured to receive an input indicative of a position of the to-be-installed lighting device with respect to the physical area, and render, on the display of the augmented reality device, a virtual representation of the to-be-installed lighting device, wherein the virtual representation is overlaid on the image of the physical area at the position. FIG. 5 illustrates an example of an augmented reality device 102 comprising an AR software application wherein two virtual representations 130, 132 are positioned as an overlay on the image of the physical area. The virtual representations 130, 132 represent the to-be-installed lighting devices. The input indicative of a position of the to-be-installed lighting device may, for example, be a user input. The user may position the virtual representation on the image, whereupon the level of disinfection of the target surface 140 may be determined based on the (new) position/location of the lighting device 130 relative to the target surface. Alternatively, the input may be a system input for automatic placement of the lighting device 130, for instance at a central location in the physical area, or above a predefined target surface 140 in the physical area 100.

As exemplified in FIG. 5, the processor 106 may be further configured to determine a further irradiation value of disinfection light provided or to be provided by a further (already installed or to-be-installed) lighting device 132 on the target surface 140. The disinfection levels provided at the subareas (squares in FIG. 5) of the target surface 140 indicate aggregated disinfection levels of both the first lighting device 130 and the further lighting device 132 at subareas illuminated by both lighting devices 130, 132. A user may provide a user input to select a lighting device 130, 132, whereupon the disinfection level of that selected lighting device is indicated on the display 110. The indicators of the different levels of disinfection may be rendered such that they are distinguishable by a user (e.g. in different colors, with different patterns, etc.), such that the effect of disinfection of both lighting devices on the target surface is clear to the user.

The processor 106 may be configured to select the to-be-installed lighting device 130 (and its virtual representation) from a plurality of lighting devices. The processor 106 may, for example, render a list of lighting devices 130 on the display 110, and the user may provide a user input via the user interface 104 to select a to-be-installed lighting device 106. Additionally or alternatively, the processor 106 may be configured to select the to-be-installed lighting device 130 based on one or more disinfection criteria. A disinfection criterium may be provided by a user via the user interface 104. Additionally or alternatively, the processor 106 may be configured to analyze sensor data of one or more sensors located in the physical area or comprised in the augmented reality device 102, and determine one or more selection criteria based on the sensor data. Additionally or alternatively, the processor 106 may be configured to analyze one or more images captured by the camera 108, and determine one or more selection criteria based on the image analysis.

A first example of a disinfection criterium may be a target disinfection level per surface area. The processor 106 may, for example, select a lighting device with a higher lumen output if a higher level of disinfection is required. A second example of a disinfection criterium may be the type of material of the target surface 140. The processor 106 may obtain information about the material of the target surface 140, for instance from a memory, from a user, from sensor data or from the image, and select a lighting device based thereon. The processor 106 may, for example, select a lighting device with a higher disinfection level for a first material (e.g. wood) compared to a second material (e.g. stainless steel). A third example of a disinfection criterium may be the type of the physical area 100 and/or the type of the target surface 140. The processor 106 may obtain information about the type, for instance from a memory, from a user, from sensor data or from the image, and select a lighting device based thereon. The processor 106 may, for example, select a lighting device with a higher disinfection level for a first type of target surface (e.g. a table) compared to a second type of target surface (e.g. a floor). The processor 106 may, for example, select a lighting device with a higher disinfection level for a first type of physical area (e.g. an operating room) compared to a second type of physical area (e.g. a hallway). Another example of a disinfection criterium may be activities that are typically performed in the physical area 100 and/or at the target surface 140. The processor 106 may obtain information about the activities, for instance from a memory, from a user, from sensor data or from the image, and select a lighting device based thereon. Another example of a disinfection criterium is a type of pathogen (e.g. virus, bacteria, mold, etc.) for which the target surface needs to be disinfected. Another example of a disinfection criterium is the power consumption for disinfecting the target surface 140.

Another example of a disinfection criterium is an expected presence of living beings in the physical area 100. For example, for 405 nm purple visible light disinfection lighting, the safety standard is based on the image on the retina, whereby the exposure duration and the radiance of the source is prescribed. For example, it is also well established that UV-C light (231-280 nm) must be shielded from humans as it poses a carcinogenic and eye safety risk, whereas for the Far UV range (200-230 nm), it has been shown that the risk is strongly reduced, since the Far UV light neither penetrates the top layer of the human skin nor the tear layer of the eye. Thus, while Far-UV-C light cannot reach or damage living human cells, it can still penetrate and kill the viruses and bacteria floating in the air as well as disinfecting surfaces. For instance, when living beings are expected to be present in the physical area 100, Far UV-C lamps that emit light with a wavelength around 222 nm may be used, or rare gas-halogen discharge light sources may be used that produce a significant emission in the Far UV-C region, e.g. in a wavelength range from 205 to 230 nm. When no living beings are exposed to the disinfection light, light sources such as pulsed xenon arc UVGI lamps emitting UV and visible radiant energy - which kill both viruses and bacteria -may be used. The pulsed xenon arc lamps may be filtered such that only the UV light used for disinfection is emitted.

The processor 106 may be further configured to obtain contextual parameters about the physical area 100 and/or the target surface 140, and determine the level of disinfection further based on the contextual parameters. The level of disinfection - indicating to what extent the target surface 140 is disinfected by the disinfection light that is emitted by the lighting device 130 - may be affected by contextual parameters. Disinfection of the target surface may be more effective under certain conditions compared to other conditions. The processor 106 may, for example, receive a user input provided by a user via the user interface 104 indicative of the contextual parameters. Additionally or alternatively, the processor 106 may be configured to analyze sensor data of one or more sensors located in the physical area 100 or comprised in the augmented reality device 102, and determine the contextual parameters based on the sensor data. Additionally or alternatively, the processor 106 may be configured to analyze one or more images captured by the camera 108, and determine/extract the contextual parameters based on the image analysis.

An example of a contextual parameter may be the type of material of the target surface. The processor 106 may obtain information about the material of the target surface 140, for instance from a memory, from a user, from sensor data or from the image, and determine the level of disinfection based thereon. The processor 106 may, for example, determine a lower disinfection level for a first material (e.g. wood) compared to a second material (e.g. stainless steel). Another example of a contextual parameter may be the type of the physical area 100 and/or the type of the target surface 140. The processor 106 may obtain information about the type, for instance from a memory, from a user, from sensor data or from the image, and determine the level of disinfection based thereon. The processor 106 may, for example, determine a higher disinfection level for a first type of target surface (e.g. a floor) compared to a second type of target surface (e.g. a table). Another example of a contextual parameter may be activities that are typically performed in the physical area 100 and/or at the target surface 140.

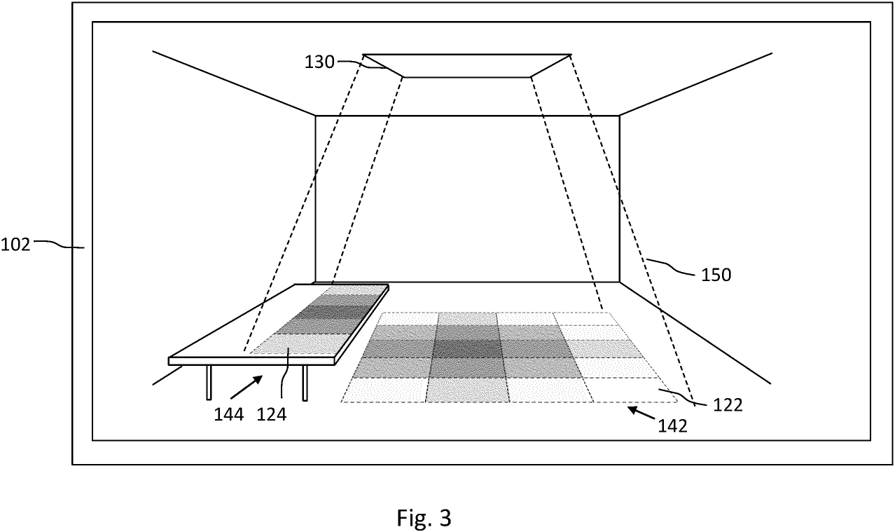

The processor 106 may be further configured to determine a second irradiation value for a second target surface in the physical area 100, and determine a second level of disinfection for the second target surface. This has been illustrated in FIG. 3, which shows an augmented reality device 102 rendering a plurality of indicators 122, 124 (squares) on respective target surfaces 142, 144. The indicators indicate the levels of disinfection for respective subareas of the target surfaces 142, 144. The levels of disinfection on the target surfaces 142, 144 are based on respective irradiation values, which are determined based on the location of the lighting device with respect to the respective target surfaces 142, 144 and based on properties of the disinfection light provided or to be provided by the lighting device 130. Respective indicators indicating the respective levels of disinfection are rendered on the display 110 of the augmented reality device 102 as an overlay on the image. This is beneficial, because the user can see the effect of the already installed/to-be-installed lighting device 130 on the different target surfaces 142, 144. Additionally, the respective levels of disinfection may be based on contextual parameters. The first target surface 142 (e.g. the floor) may for example require less UV light for disinfection and may therefore have a higher level of disinfection, whereas the second target surface (e.g. the table) may require more UV light for disinfection and may therefore have a lower level of disinfection.

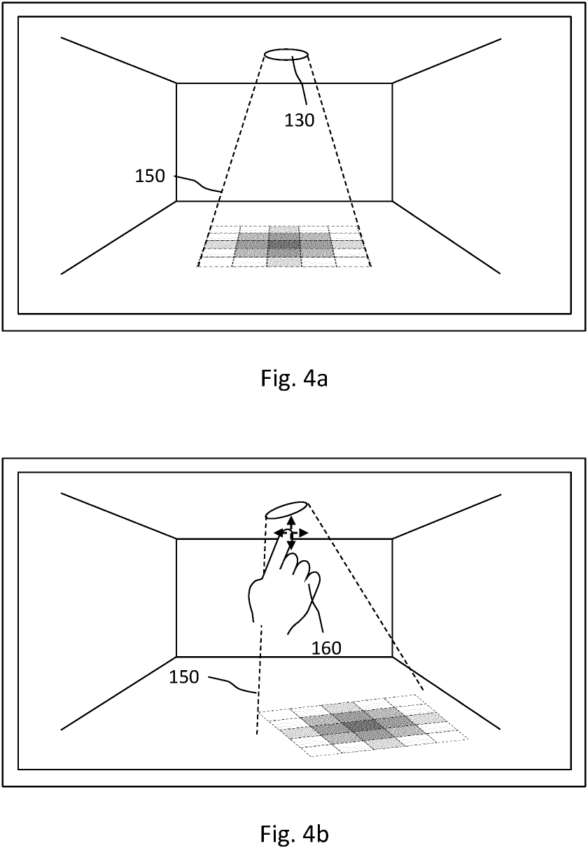

The beam of disinfection light (of an already installed) lighting device 130 may be adjustable. The lighting device 130 may, for example, comprise actuators for changing the orientation of the lighting device and/or controllable/adjustable optics for changing the beam shape and/or direction. Additionally or alternatively, the lighting device may comprise an array of individually controllable (LED) lighting units configured to be controlled to change the beam shape and/or direction. Lighting devices 130 with adjustable beams are known in the art and will therefore not be discussed in detail. The processor 106 may be configured to receive an input indicative of an adjustment of the beam of disinfection light of the lighting device, and to control the lighting device 130 to adjust the beam of disinfection light of the lighting device 130 based on the input, for instance by communicating lighting control signals to the lighting device 130 via the communication unit. The shape and/or the direction of the beam may be adjustable. The user may, for example, provide a touch input 160 on a touch screen of the augmented reality device to change the orientation (direction) of the beam, or to change the shape of the beam (see FIGS. 4a and 4b, wherein a user provides a user input to change the orientation of the beam). Alternatively, the input may be a system input for automatic adjustment of the beam, for example based on one or more contextual parameters as described above. When the target surface 140 has been defined, the processor 106 may adjust the beam shape to increase or decrease the disinfection level of the target surface 140, for instance by adjusting the beam such that the beam illuminates the target surface 140 primarily/only. If, for example, the target surface is a table, the beam shape may be adjusted such that the lighting device 130 primarily/only illuminates the table.

The processor 106 may be further configured to render, on the display 110 of the augmented reality device 102, a beam shape indicator 150 overlaid on the image of the physical area 100, wherein the beam shape indicator 150 indicates the shape of the beam of the disinfection light. The beam shape may be circular, oval, substantially rectangular, symmetrical, asymmetrical, etc. The shape of the beam of the disinfection light may, for example, be determined based on the lighting data/photometric data of the lighting device 130.

The processor 106 may be further configured to render, on the display 110 of the augmented reality device 102, a photodegrading indicator overlaid on the image of the physical area 100. The photodegrading indicator may indicate the effect of the chosen disinfection lighting on equipment and furnishing within the room that are not resistant to the disinfection light. For instance, materials with fugitive pigments, such as organically dyed textiles of furniture, are known to fade or discolor under UV light. Additionally or alternatively, the processor 106 may be further configured to render, on the display 110 of the augmented reality device 102, a visual appearance indicator overlaid on the image of the physical area 100. The visual appearance indicator may indicate how the appearance objects/surfaces changes due to the disinfection light, or it may indicate to what extent the visual appearance changes. This may be based on the contextual parameters of the target surface 140 such as the material of the target surface 140/object. For example, UV light can induce fluorescence (glowing) from some materials, particularly fabrics that have been washed in detergents containing bluing agents. Similarly, for continuous purple-light disinfection the appearance of objects in the space will be impacted due to the blue spectral component (20% of the white light spectrum being concentrated around 405 nm).

The processor 106 may be further configured to receive user input (e.g. via the user interface 104) indicative of an adjustment of the properties of the disinfection light provided or to be provided by the lighting device, and to adjust the properties of the disinfection light based on the user input. If the lighting device 130 has already been installed in the physical area 100, the processor 106 may communicate a lighting control command to the lighting device 130 to change the lighting device’s light emission properties, for example the intensity, the spectrum of the emitted light, etc. If the lighting device 130 is yet to be installed, the processor 106 may adjust a virtual representation of the light emission of the virtual lighting device 130. After the properties have been adjusted, the processor 106 may determine a new irradiation value based on the new properties and therewith a new level of disinfection of the target surface 140 based on the new irradiation value. The processor 106 may then update the indicator 120 based on the new level of disinfection.

The processor 106 may be further configured to determine a period of time that a person, animal or an object can safely be exposed to the disinfection light, and render a safety indicator indicative of the period of time as an overlay on the image on the display 110. The processor 106 may be configured to determine the period of time based on the type of person (e.g. based on the person’s skin color, amount of clothing, height of a person’s eyes with respect to the target surface 140, etc.), based on the type of animal and/or based on the type/material of the object (for some objects/materials disinfection light is more detrimental than others). The processor 106 may, for example, access a database storing a lookup table comprising associations between persons, animals or objects and one or more irradiation values of the lighting device 130, and determine the period of time based thereon. If the user changes one of the properties of the UV light, the period of time and therewith the safety indicator may be updated by the processor 106. The processor 106 may render different safety indicators for different subareas of the target surface 140. For instance, the darker gray areas in FIG. 2 may be indicative of a shorter period of time that the person/animal/object can safely be exposed to the disinfection light, and the light gray areas in FIG. 2 may be indicative of a longer period of time that the person/animal/object can safely be exposed to the disinfection light.

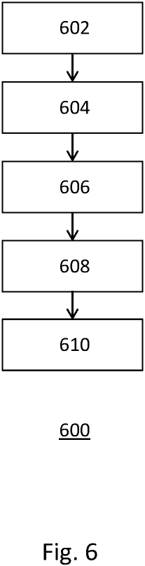

FIG. 6 shows schematically a method of determining disinfection levels caused by a lighting device, the lighting device being configured to emit disinfection light. The method comprises:
capturing 602, by a camera of an augmented reality device, an image of a physical area,
displaying 604, on a display of the augmented reality device, the image,
determining 606 an irradiation value of disinfection light provided or to be provided by the lighting device on the target surface, wherein the irradiation value is determined based on a location of the lighting device with respect to the target surface and based on properties of the disinfection light provided or to be provided by the lighting device,
determining 608 a level of disinfection of the target surface based on the irradiation value of disinfection light, and
rendering 610, on the display of the augmented reality device, an indicator indicating the level of disinfection, wherein the indicator is overlaid on the image of the physical area. The method 600 may be executed by computer program code of a computer program product when the computer program product is run on a processing unit of a computing device, such as the processor 106 of the augmented reality device 102.

It should be noted that the above-mentioned embodiments illustrate rather than limit the invention, and that those skilled in the art will be able to design many alternative embodiments without departing from the scope of the appended claims.

In the claims, any reference signs placed between parentheses shall not be construed as limiting the claim. Use of the verb “comprise” and its conjugations does not exclude the presence of elements or steps other than those stated in a claim. The article “a” or “an” preceding an element does not exclude the presence of a plurality of such elements. The invention may be implemented by means of hardware comprising several distinct elements, and by means of a suitably programmed computer or processing unit. In the device claim enumerating several means, several of these means may be embodied by one and the same item of hardware. The mere fact that certain measures are recited in mutually different dependent claims does not indicate that a combination of these measures cannot be used to advantage.

Aspects of the invention may be implemented in a computer program product, which may be a collection of computer program instructions stored on a computer readable storage device which may be executed by a computer. The instructions of the present invention may be in any interpretable or executable code mechanism, including but not limited to scripts, interpretable programs, dynamic link libraries (DLLs) or Java classes. The instructions can be provided as complete executable programs, partial executable programs, as modifications to existing programs (e.g. updates) or extensions for existing programs (e.g. plugins). Moreover, parts of the processing of the present invention may be distributed over multiple computers or processors or even the ‘cloud’.

Storage media suitable for storing computer program instructions include all forms of nonvolatile memory, including but not limited to EPROM, EEPROM and flash memory devices, magnetic disks such as the internal and external hard disk drives, removable disks and CD-ROM disks. The computer program product may be distributed on such a storage medium, or may be offered for download through HTTP, FTP, email or through a server connected to a network such as the Internet.

1. A method of determining disinfection levels caused by a lighting device installed in or to be installed in a physical area, the lighting device being configured to emit disinfection light, wherein the disinfection light is ultraviolet light, the method comprising:
capturing, by a camera of an augmented reality device, an image of the physical area,
displaying, on a display of the augmented reality device, the image,
determining, by a processor, an irradiation value of disinfection light provided or to be provided by the lighting device on a target surface, wherein the irradiation value is determined based on a location of the lighting device with respect to the target surface and based on properties of the disinfection light provided or to be provided by the lighting device,
determining, by the processor, a level of disinfection of the target surface based on the irradiation value of disinfection light, wherein the level of disinfection is indicative of an exposure time required to disinfect the target surface with the disinfection light , and
rendering, on the display of the augmented reality device, an indicator indicating the level of disinfection, wherein the indicator is overlaid on the image of the physical area, and wherein the indicator indicates the exposure time.

2. The method of claim 1, wherein the lighting device has been installed in the physical area.

3. The method of claim 2, wherein the beam of disinfection light of the lighting device is adjustable, and wherein the method further comprises:
receiving input indicative of an adjustment of the beam of disinfection light of the lighting device and
adjusting the beam of disinfection light of the lighting device based on the input.

4. The method of claim 1, wherein the lighting device is a to-be-installed lighting device, and wherein the method further comprises:
receiving an input indicative of a position of the to-be-installed lighting device with respect to the physical area,
rendering, on the display of the augmented reality device, a virtual representation of the to-be-installed lighting device, wherein the virtual representation is overlaid on the image of the physical area at the position.

5. The method of claim 4, further comprising the step of:
selecting the lighting device from a plurality of lighting devices based on one or more disinfection criteria.

6. The method of claim 5, wherein the method further comprises:
receiving user input indicative of the one or more disinfection criteria.

7. The method of claim 1, further comprising:
obtaining contextual parameters about the physical area and/or the target surface, and
determining the level of disinfection further based on the contextual parameters.

8. The method of claim 7, wherein the method further comprises:
receiving user input indicative of the contextual parameters.

9. The method of claim 1, further comprising:
determining a second irradiation value of disinfection light provided or to be provided by the lighting device on a second target surface in the physical area, wherein the second irradiation value is determined based on the location of the lighting device with respect to the second target surface and based on properties of the disinfection light provided or to be provided by the lighting device,
determining a second level of disinfection of the second target surface based on the second irradiation value of disinfection light, and
rendering, on the displaying of the augmented reality device, a second indicator indicating the second level of disinfection, wherein the second indicator is overlaid on the image of the physical area.

10. The method of claim 1, further comprising:
determining a further irradiation value of disinfection light provided or to be provided by a further lighting device on the target surface, wherein the further lighting device has been installed in the physical area, and wherein the further irradiation value is determined based on a location of the further lighting device with respect to the target surface and based on properties of the disinfection light provided or to be provided by the further lighting device,
determining a further level of disinfection of the target surface based on the further irradiation value of disinfection light, and
rendering, on the display of the augmented reality device, a further indicator indicating the further level of disinfection, wherein the further indicator is overlaid on the image of the physical area.

11. The method of claim 1, further comprising:
determining a period of time that a person, animal or an object can safely be exposed to the disinfection light, and
rendering a safety indicator indicative of the period of time.

12. The method of claim 1, further comprising:
rendering, on the display of the augmented reality device, a beam shape indicator overlaid on the image of the physical area, wherein the beam shape indicator indicates the shape of the beam of the disinfection light.

13. The method of claim 1, further comprising:
receiving user input indicative of an adjustment of the properties of the disinfection light provided or to be provided by the lighting device,
adjusting the properties of the disinfection light based on the user input.

14. A computer program product for a computing device, the computer program product comprising computer program code to perform the method of claim 1 when the computer program product is run on a processing unit of the computing device.

15. An augmented reality device for determining disinfection levels caused by a lighting device installed in or to be installed in a physical area, the lighting device being configured to emit disinfection light, wherein the disinfection light is ultraviolet light, the augmented reality device comprising:
a camera configured to capture an image of the physical area,
a display configured to display the image,
a processor configured to:
determine an irradiation value of disinfection light provided or to be provided by the lighting device on the target surface, wherein the irradiation value is determined based on a location of the lighting device with respect to the target surface and based on properties of the disinfection light provided or to be provided by the lighting device,
determine a level of disinfection of the target surface based on the irradiation value of disinfection light, wherein the level of disinfection is indicative of an exposure time required to disinfect the target surface with the disinfection light, and
render, on the display, an indicator indicating the level of disinfection, wherein the indicator is overlaid on the image of the physical area, and wherein the indicator indicates the exposure time.
